(19) United States
(12) Patent Application Publication
(10) Pub. No.: US 2010/0042164 A1
(43) Pub. Date: Feb. 18, 2010

(54) Expansion screw set and hollow nail and interior nail thereof

(76) Inventors: Shih-Tseng Lee, Taipei (TW); Jyi-Feng Chen, Xindian City (TW); Chieh-Tsai Wu, Linkou Shiang (TW); Hsun-Hui Hsu, Taipei (TW); Nan-Chun Liu, Taishan Shiang (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/588,563

(22) Filed: Oct. 20, 2009

Related U.S. Application Data
(62) Division of application No. 11/723,425, filed on Mar. 20, 2007.

(30) Foreign Application Priority Data
Aug. 10, 2006 (TW) 095214095

(51) Int. Cl. A61B 17/86; A61B 17/20

(52) U.S. Cl. 606/304; 604/22

(57) ABSTRACT

The invention provides an expansion screw set, a hollow nail and an interior nail. The expansion screw set is composed of the hollow nail and the interior nail. The hollow nail has a hollow structure. A first thread and a second thread are provided for the inside and the outside of the hollow nail. The interior nail has a third thread. The hollow nail could nail an object through the first thread. The third thread of the interior nail corresponds to the second thread of the hollow nail. Therefore, the interior nail can be fastened into the hollow structure after the interior nail is nailed into the hollow nail. Moreover, the hollow nail has a separation line which runs parallel to the penetration direction of the hollow structure. Alternatively, the hollow nail has a plurality of penetration holes which is vertical to the penetration direction of the hollow structure. After the interior nail is nailed into the hollow structure, the separation line can be stretched out through the interior nail. Alternatively, contents within the hollow structure are squeezed to the outside from the penetration holes through the interior nail.

### RELATED APPLICATIONS

This application is a Divisional patent application of co-pending application Ser. No. 11/723,425, filed on 20 Mar. 2007. The entire disclosure of the prior application Ser. No. 11/723,425 from which an oath or declaration is supplied, is considered a part of the disclosure of the accompanying Divisional application and is hereby incorporated by reference.

### BACKGROUND OF INVENTION

1. Field of the Invention

The present invention relates generally to an expansion screw set with a hollow nail and an interior nail, and more particularly to the expansion screw set that increases the fastening performance by combining the hollow nail and the interior nail.

2. Description of Related Art

According to commonly used fracture treatment or prevention technologies, a bone plate is nailed securely into a bone through a plurality of bone nails. Currently, most of bone nails for medical purpose are solid structures without surface holes, and only threads on the surfaces of the bone nails are fixed into the bone, so the fastness degree of the bone nails may not be secured into the bone completely and it will depend upon the bone density. However, the bone nails and the bone plates likely get loose due to weights, the increase of the bone, osteoporosis or other factors of the patients after surgical operation is completed.

For this reason, bone cement is often poured into the bone, and then bone nails are nailed through the bone plates into the bone. When the threads on the surface of the bone nail are fixed into the bone, the clearance is filled with the bone cement for tightly fixing the bone nails and the loose bone. However, the body of the bone nail may not fully drill into the bone due to the hardening characteristics of the bone cement, or even damage the hardened bone cement, leading to the loosening of bone nails from bone plates.

Thus, to overcome the aforementioned problems of the prior art, it would be an advancement if the art to provide an improved structure that can significantly improve the efficacy.

In view of shortcomings of the prior art, the inventor of the present invention based on years of experience in the related industry to conduct extensive researches and experiments, and finally developed an expansion screw set with a hollow nail and an interior nail in accordance with the present invention to overcome the aforementioned shortcomings.

### SUMMARY OF THE INVENTION

The purpose of the present invention is to provide an expansion screw set with a hollow nail and an interior nail, and more particularly to the expansion screw set that increases the fastening performance by combining the hollow nail and the interior nail in order to resolve the loosening problem and provide additional functions through the bone cement or other agents.

For the above-specified purposes, the expansion screw set of the present invention is used to nail bones, and is composed of a hollow nail and an interior nail. The hollow nail has a hollow structure, and also has some threads on the surface to secure the bones. Moreover, the hollow nail has through holes or a separation line, of which the through holes are vertical to the penetration direction of the hollow structure, and the separation line is located at the bottom of hollow nail in parallel to the penetration direction of the hollow structure. After the interior nail is nailed into the hollow structure of the hollow nail, the contents within the hollow structure then are squeezed to the outside, or the separation line of the hollow nail is pressed.

The hollow structure preferably contains liquid or semisolid substances such as bone cement, marrow, artificial marrow, bone slurry or nutrient. If the interior nail is nailed into the hollow structure, the liquid or semisolid substances can be squeezed to the outside of the hollow structure through the through hole, thereby improving the fastening or treatment effect. On the other hand, if the interior nail is fully drilled into the hollow structure, and the external diameter of the bottom of the interior nail is bigger than the internal diameter of the bottom of the hollow structure, the separation line of the hollow nail can be pressed such that the hollow nail is securely fastened. Moreover, the hollow structure and the interior nail have threads corresponding to each other. When the interior nail drills into the hollow structure, the interior nail can be fixed into the hollow structure.

### BRIEF DESCRIPTION OF THE DRAWINGS

FIG. 1 shows a cross-sectional view of a hollow nail of the preferred embodiment of the present invention.

FIG. 2 shows a perspective view of a hollow nail of the preferred embodiment of the present invention.

FIG. 3 shows a cross-sectional view of the first embodiment of an interior nail of the present invention.

FIG. 4 shows a perspective view of the first embodiment of an interior nail of the present invention.

FIG. 5 shows a cross-sectional view of the first embodiment of an expansion screw set of the present invention.

FIG. 6 shows a cross-sectional view of the second embodiment of an interior nail of the present invention.

FIG. 7 shows a perspective view of the second embodiment of an interior nail of the present invention.

FIG. 8 shows a cross-sectional view of the second embodiment of an expansion screw set of the present invention.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings.

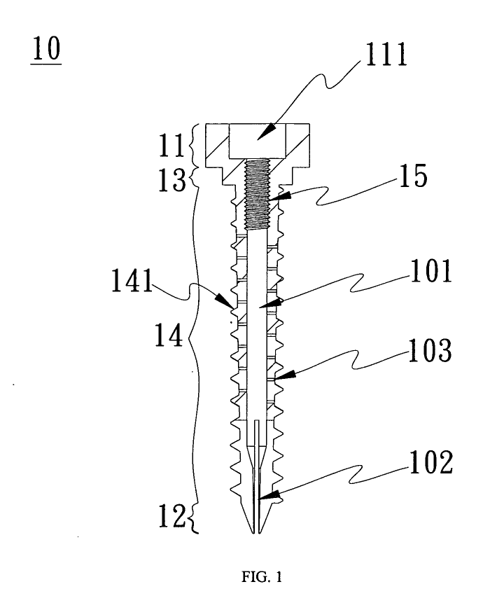

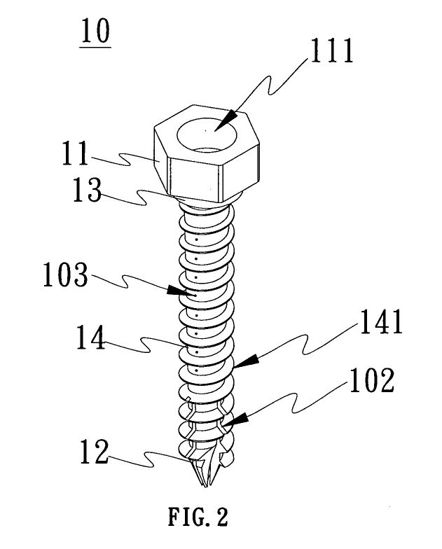

FIGS. 1 and 2 depict a cross-sectional view and a perspective view of a hollow nail of the preferred embodiment of the present invention. The hollow nail 10 comprises a head 11, a neck 13, a rod body 14 and a nail portion 12. The hollow nail 10 has a hollow structure 101, which penetrates the head 11, the neck 13, the rod body 14 and the nail portion 12. The head 11 is provided with a circular groove 111, while the external diameter of the neck 13 is between the external diameter of the head 11 and the external diameter of the first thread 141 of the rod portion 14, and the neck 13 could further provide a stress for the surface of an object. The surface of the rod body 14 has a first thread 141 and a plurality of penetration holes 103, while the external diameter of the nail portion 12 is reduced from top to bottom. Moreover, a plurality of separation lines 102 is arranged from the nail portion 12 to the bottom of the rod body 14. In the hollow structure 101, a second thread 15 is extended from the bottom of the circular groove 111 of the head 11 to the upper part of the rod body 14. The head 11 is generally designed into a hexagonal shape, which can apply forces to rotate the hollow nail 10 such that the hollow nail 10 penetrates a through hole of a fastening plate for fastening purpose. The penetration direction of the hollow structure 101 runs parallel to the separation line 102, and perpendicular to the penetration hole 103. The neck 13 is preferably designed into a cylinder. The object is preferably the bone. The bone cement, marrow, artificial marrow, bone slurry or nutrition are pre-injected into the hollow structure 101. Moreover, the neck 13 may provide the hollow nail with a certain degree of flexibility in relation to the fastening plate.

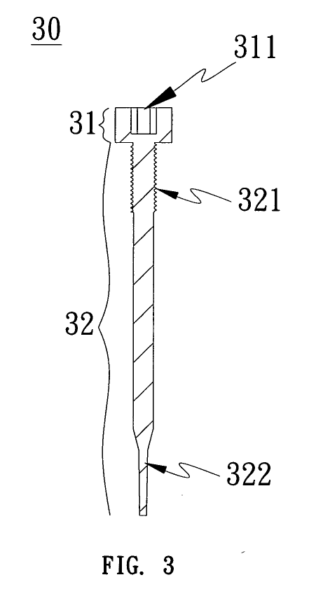

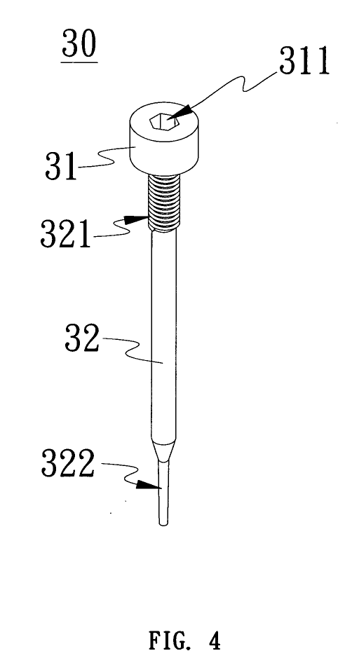

FIGS. 3 and 4 depict a cross-sectional view and a perspective view of the first embodiment of the interior nail of the present invention. The interior nail 30 comprises a nail head 31 and a nail body 32. The nail head 31 generally has a hexagonal groove 311 for rotation downwards by adding an external force. The nail body 32 has a third thread 321, and the external diameter of a lower end 322 of the nail body 32 is smaller. The nail head 31 can be accommodated within the circular groove 111 of the head 11 of the aforementioned hollow nail 10. The third thread 321 corresponds to the second thread 15 within the hollow structure 101 of the hollow nail 10, while the nail body 32 and its lower end 322 correspond to the hollow structure 101 of the hollow nail 10 so that the interior nail 30 can be securely fastened into the hollow nail 10.

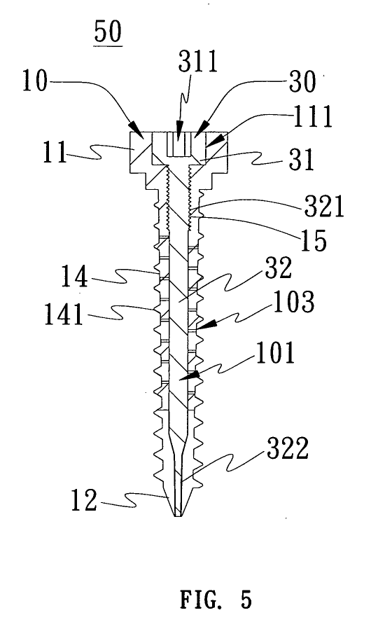

FIG. 5 depicts the cross-sectional view of the first embodiment of an expansion screw set of the present invention. The expansion screw set 50 comprises a hollow nail 10 and an interior nail 30. The nail head 31 of the interior nail 30 is accommodated within the circular groove 111 of the hollow nail 10, while the second thread 15 of the hollow nail 10 is tightly combined to the third thread 321 of the interior nail 30. Moreover, the nail body 32 of the interior nail 30 is accommodated within the hollow structure 101 of the hollow nail 10. When the hollow nail 10 penetrates the through-hole of the fastening plate, and fastens an object via the first thread 141, the liquid or semisolid substances can be filled into the hollow structure 101, and then the interior nail 30 is drilled into the hollow nail 10. Since the hollow structure 101 corresponds to the nail body 32, the contents in the hollow structure are squeezed by the interior nail 30 to the outside from the penetration hole 103, thereby increasing the fastening or treatment efficiency of the expansion screw set 50. The object is preferably a bone. The above-specified contents are generally liquid or semisolid substances, such as bone cement, marrow, artificial marrow, bone slurry or nutrition.

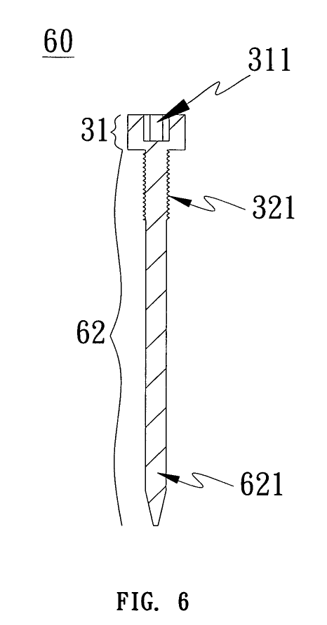

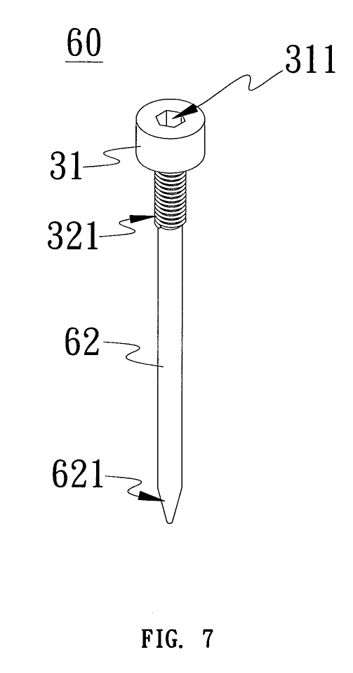

FIGS. 6 and 7 depict a cross-sectional view and a perspective view of the second embodiment of the interior nail of the present invention. The interior nail 60 comprises a nail head 31 and a nail body 62. The nail head 31 generally has a hexagonal groove 311 for rotation downwards by adding an external force. The nail body 62 has a third thread 321 and a lower end 621 with a nail-like shape. The nail head 31 can be accommodated within the circular groove 111 of the head 11 of the aforementioned hollow nail 10. The third thread 321 corresponds to the second thread 15 in the hollow structure 101 of the hollow nail 10. The nail body 62 can be accommodated in the hollow structure 101, and the lower end 621 of the nail body 60 can drill into the hollow structure 101 of the nail portion 12 of the hollow nail 10 so that the interior nail 60 can be tightly fastened into the hollow nail 10.

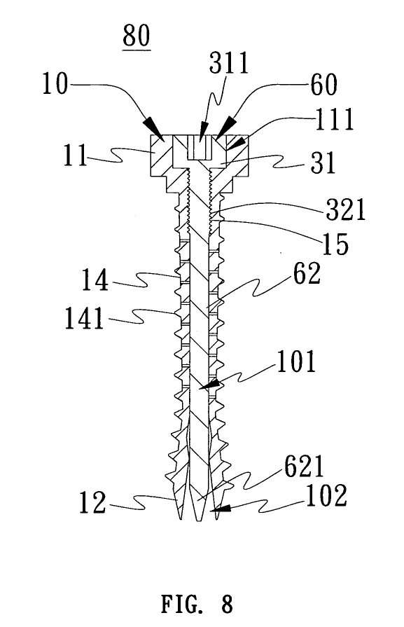

FIG. 8 depicts the sectional view of the second embodiment of expansion screw set of the present invention. The expansion screw set 80 comprising a hollow nail 10 and an interior nail 60. The nail head 31 of the interior nail 60 is accommodated within the circular groove 111 of the hollow nail 10, while the second thread 15 of the hollow nail 10 is tightly combined to the third thread 321 of the interior nail 60. Since the external diameter of the lower end 621 of the nail body 62 is bigger than the internal diameters of the bottom of the rod body 14 of the hollow nail 10 and the nail portion 12, the separation line 102 of the hollow nail 10 can be thus stretched out. Therefore, when the hollow nail 10 penetrates the through-hole of the fastening plate, and fastens contents via the first thread 141, the interior nail 60 can be drilled into the hollow nail 10. As the nail body 32 can drill into the hollow structure 101 to stretch out the separation line 102 of the hollow nail 10, thus improving the fastening effect of the expansion screw set 80. The above-specified contents are generally liquid or semisolid substances, such as bone cement, marrow, artificial marrow, bone slurry or nutrition.

Although the invention has been explained in relation to its preferred embodiment, it is to be understood that many other possible modifications and variations can be made without departing from the spirit and scope of the invention as hereinafter claimed.

1. An expansion screw set for fastening an object, comprising:
a hollow nail having a hollow structure, and a surface of the hollow nail having a thread for fastening the object; and
an interior nail for drilling into the hollow structure to squeeze contents in the hollow structure and expand a lower portion of the hollow nail outwardly;
wherein the lower portion of the hollow nail is provided with at least a separation line, which runs parallel to the penetration direction of the hollow structure and the lower portion is expanded outwardly from the separation line by the interior nail when the interior nail fully drills into the hollow structure.

2. The expansion screw set defined in claim 1, wherein the hollow nail has at least a penetration hole, and the penetration direction of the penetration hole runs perpendicular to the penetration direction of the hollow structure.

3. The expansion screw set defined in claim 1, wherein the contents in the hollow structure are a liquid or a semisolid substance.

4. The expansion screw set defined in claim 2, wherein the interior nail drills into the hollow structure, and the contents within the hollow structure are squeezed to the outside from the penetration holes.

5. The expansion screw set defined in claim 3, wherein the liquid or semisolid substance is bone cement, marrow, artificial marrow, bone slurry or nutrition.

6. The expansion screw set defined in claim 1, wherein a plurality of threads on a surface of the hollow structure and a surface of the interior nail correspond to each other, and when the interior nail drills into the hollow structure, the interior nail is fastened.

7. The expansion screw set defined in claim 1, wherein the object is a bone.

8. The expansion screw set defined in claim 1, wherein the hollow nail comprises a neck for providing a stress for the surface of the object.

9. A hollow nail applied to an expansion screw set to lock an object, the hollow nail comprising:
a hollow structure for providing a rod body of the expansion screw set to drill;
a first thread disposed to a surface of the hollow nail to lock the object; and
a second thread disposed to the hollow structure, the second thread corresponding to a shape of the rod body to fasten the rod body within the hollow structure;
wherein the rod body defines an interior nail, a lower portion of hollow nail has at least one separation line which runs parallel to the penetration direction of the hollow structure, and the lower portion is expanded outwardly from the separation line by the interior nail when the interior nail fully drills into the hollow structure.

10. The hollow nail defined in claim 9, wherein the hollow nail has at least a penetration hole which runs perpendicular to the penetration direction of the hollow structure.

11. The hollow nail defined in claim 10, wherein a liquid or a semisolid substance is injected into the hollow structure.

12. The hollow nail defined in claim 11, wherein the liquid or semisolid substance is squeezed to the outside from the hollow structure through the penetration hole when the interior nail is drilled into the hollow structure.

13. The hollow nail defined in claim 12, wherein the liquid or semisolid substance is bone cement, marrow, artificial marrow, bone slurry or nutrition.

14. The hollow nail defined in claim 9, wherein the object is a bone.

15. The hollow nail defined in claim 9, wherein the hollow nail further comprises a neck for providing a stress for the surface of the object.

16. An interior nail applied in an expansion screw set to lock an object, the interior nail comprising:
a rod body;
a nail portion being a nail shape, and for connecting a lower end of the rod body; and
a third thread disposed to the rod body, and corresponding to a hollow structure of the expansion screw set;
wherein the third thread enables the rod body and the nail portion to drill and fasten into the hollow structure;
wherein the hollow structure is provided from a hollow nail of the expansion screw set, a lower portion of the hollow nail has at least one separation line which runs parallel to the penetration direction of the hollow structure, and when the interior nail fully drills into the hollow structure, the lower portion is expanded outwardly from the separation line by the interior nail.

17. The interior nail defined in claim 16, wherein the hollow nail has at least a penetration hole which runs perpendicular to the penetration direction of the hollow structure.

18. The interior nail defined in claim 16, wherein a liquid or a semisolid substance is injected into the hollow structure.

19. The interior nail defined in claim 18, wherein the liquid or semisolid substance is squeezed to the outside from the hollow structure through the penetration hole when the interior nail drills into the hollow structure.

20. The interior nail defined in claim 18, wherein the liquid or semisolid substance is bone cement, marrow, artificial marrow, bone slurry or nutrition.
